(19) United States
(12) Patent Application Publication
(10) Pub. No.: US 2013/0178757 A1
(43) Pub. Date: Jul. 11, 2013

(54) FUNCTIONAL EEG IMAGER

(71) Applicants: Michael Linderman, Ogdensburg, NY (US); Valery I. Rupasov, Gouverneur, NY (US)

(72) Inventors: Michael Linderman, Ogdensburg, NY (US); Valery I. Rupasov, Gouverneur, NY (US)

(73) Assignee: Norconnect Inc., Ogdensburg, NY (US)

(21) Appl. No.: 13/781,828

(22) Filed: Mar. 1, 2013

Related U.S. Application Data
(63) Continuation-in-part of application No. 13/341,465, filed on Dec. 30, 2011.
(60) Provisional application No. 61/607,708, filed on Mar. 7, 2012.

(51) Int. Cl. A61B 5/0484; A61B 5/00; A61B 5/04

(52) U.S. Cl. CPC A61B 5/0484; A61B 5/04012; A61B 5/742; A61B 5/4064
USPC 600/544

(57) ABSTRACT

A system for identifying the connectivity between different brain regions to determine the functional role of brain regions in various human and animal actions.

### CROSS-REFERENCE TO RELATED APPLICATION

This application claims the benefit of U.S. Provisional Patent Application No. 61/607708 filed Mar. 7, 2012.

This application is a continuation-in-part of U.S. patent application Ser. No. 13/341,465 filed Dec. 30, 2011.

The aforementioned provisional applications disclosures are incorporated herein by reference in their entirety.

### BACKGROUND OF THE INVENTION

1. Field of the Invention

This invention relates to a system and method for studying functional connectivity between different regions of the brain using Electroencephalography (EEG). The proposed technology is based on a statistical analysis of EEG signals recorded with a standard EEG recording system. EEG data are collected while a subject performs repeatedly a set of identical actions, or trials. The EEG signals corresponding to this set of trials are then treated as a statistical ensemble of “identical systems”. Time-dependent correlation functions between neural signals are computed from the statistical ensemble of trials. The proposed statistical approach enables i) monitoring dynamical brain activity, and ii) determining dynamical functional associations between various brain areas during motor actions and passive responses to sensory stimulation. The functional EEG imager will record brain activity related to human or animal responses to typical stimuli, such as visual and auditory stimuli. This technology will allow us to visualize brain processing as images and videos and to determine the functional role of brain circuitry for various human or animal actions. Moreover, we expect that the proposed EEG imager will also serve as a powerful medical tool for early diagnostics of neurological conditions and for monitoring patient's recovery and drug efficiency.

2. Background of the Invention

At present, the only device on the market that can show dynamic functional changes is functional Magnetic Resonance Imager (fMRI). Nevertheless, this instrument has several important disadvantages stemming from the fluidal fundamentals of fMRI:
It tracks the blood flow and the status of oxygen in hemoglobin that is indirect to the electrical activity of the brain;
Therefore, functional analysis of brain activity with fMRI is limited by the characteristic time of hemodynamics, that is several seconds, which is too long for monitoring rapid changes in brain activity that happen every millisecond;
In addition, NMI recordings are extremely expensive, and many researchers and clinicians do not have access to this equipment;
fMRI system is very large and complex, and requires well-trained personnel to operate and maintain the system;
Potential health hazard exists due to intense magnetic fields.

The invented EEG-based imaging technology offers remarkable competitive advantages to medical practitioners and researchers which cannot be achieved with NMI technology:
It monitors directly electrical activity of the brain regions;
Time resolution is ten(s). of milliseconds, which captures rapid brain modulations;
The proposed technology is cost-effective, and can be broadly employed for numerous applications in both research laboratories and clinics;
The system can use standard EEG recording devices which are currently available in many research and clinical laboratories and do not require prolonged training of the personnel to operate and maintain;
EEG systems do not adversely affect human subjects, because EEG systems only record electrical signals naturally existing on the scalp surface owing, to brain. activity.

A simple schematic of the proposed multi-functional EEG imager is illustrated in FIG. 1. Here a stimulus (e.g., light or sound) initiates brain processing (visual or auditory) which is reflected in EEG changes. A computer algorithm first divides the EEG records into trials, i.e., the epochs during which the subject performs repetitive actions triggered by sensory stimuli. The statistical ensemble of trials is then characterized by time-dependent correlation functions, which quantify dynamical activity of brain areas. Using this approach, one can map dynamical functional connectivity between different brain areas during various brain responses. Brain modulations and the strength of functional connectivity between various brain areas can be presented graphically as color-coded images of the time-dependent correlation functions.

Since the first publication by D. Walter [D. O. Walter, Spectral analysis for electroencephalograms: mathematical determination of neurophysiological relationships from records of limited duration, Exp. Neural. 8, 1.55 (1963)], the coherence method, developed for the analysis of stationary random data in linear systems, has been employed in hundreds of papers dealing with the analysis of neural signals such as EEGs. In these publications, the level of coherence was used as a measure of coupling between the processes generating neural signals and of the functional association between neuronal structures [D. 0. Waiter, Coherence as a measure of relationship between EEG records, Electroencephologr. Clin. Neurophysiol. 24, 282 (1968); J. R. Rosenberg, A. M. Amjad, P. Breeze, ft R. Brillinger, and D. M. Halliday, The Fourier approach to the identification of functional coupling between neuronal spike trains, Prog. Biophys. Molec. Biol. 53, 1 (1989); P. Nunez, Neocortical Dynamics and Human EEG Rhythms (Oxford University Press, Boston, 1994); T. Mima and Mark Hallett, Electroencephalographic analysis of cortico-muscular coherence: reference effect, volume conduction and generator mechanism, Clinical Neurophysiology 110, 1892 (1999)]. The coherence method and its numerous modifications work in the frequency domain, limiting the analysis of dynamical changes in cortical activity.

Recently, we proposed and discussed [V. I. Rupasov, M. A. Lebedev, J. S. Erlichman, S. L. Lee, J. C. Leiter, and M. Linderman, Time-dependent statistical and correlation properties of neural signals during handwriting PLoS ONE 7(9): e43945] an alternative approach to the search for dynamical relationship between neural signals. In this approach, which is broadly employed, in statistics and, in particular, in statistical physics, a relationship between two random time-dependent signals x(t) and y(t) is determined by the time-dependent correlation function

C(t₁,t₂)=∫dxdy[x(t₁)−μₓ(t₁)]·[y(t₂)−μ_{y}(t₂)]p(x,y). (2)

Here, p(x,y) is the joint probability density function of two random variables, and μₓ and μ_{y} are the corresponding mean values, μₓ(t)=∫x(t)p(x)dx and μ_{y}(t)=∫y(t)p(y)dy, where p(x) is the probability density function It should be emphasized that for nonstationary EEG signals, the time dependence of correlation functions is determined not only by the time dependencies of the signals themselves, but also by the time dependencies of the. probability density functions.

For two independent random variables, the joint probability density function is factorized, that is p(x,y)˜p(x)·p(y), and the correlation function C vanishes.

The probability density functions of neural signals are not known a priori. Therefore, one needs to have a sufficiently large statistical ensemble of neural signals {xⱼ(t)} and {yⱼ(t)}, (j=1÷N) recorded during N epochs—in our case, trials during which a subject repeatedly performs an identical task—in order to apply this statistical method. in this approach, the integration with the joint probability density function in Eq. (2) is replaced by an ensemble averaging over many trials:

$$C ( {t}_{1} , {t}_{2} ) = \frac{1}{N} {\sum }_{j = 1}^{N} [ {x}_{j} ( {t}_{1} ) - {μ}_{x} ( {t}_{1} ) ] \cdot [ {y}_{j} ( {t}_{2} ) - {μ}_{y} ( {t}_{2} ) ] . \qquad ( 3 )$$

In our experiments on handwriting [V. I. Rupasov M. A. Lebedev, J. S. Erlichman, S. L. Lee, J. C. Leiter, and M. Linderman, Time-dependent statistical and correlation properties of neural signals during handwriting, PLoS ONE 7(9): e43945], the number of trials was about 400, and we used Fisher's theorem [R. M. Feldman and C. Valdez-Flores Applied Probability and Stochastic Processes (Springer, 2010)] to compute 95% confidence interval for the correlation functions. Based on this research, we expect that 100 trials (N=100) will be sufficient to compute statistically significant correlation functions.

In contrast to the coherence methods used to study the relationship between neural signals in the frequency domain, the proposed method enables to study the statistical and correlation properties of neural signals in the original time domain. That allows one to elucidate the dynamics of cortical patterns across various cortical areas and the dynamics of functional associations between different areas.

Although the neuronal signals are recorded in a wide spectral range from a few Hz to 450 Hz, the whole spectral range can he divided into more narrow spectral ranges, e.g., alpha (8-13 Hz), beta (13-30 Hz) and gamma (30-100 Hz) that enables to derive more detailed dynamical picture of neuronal activity.

In addition to a provisional patent application No. 61/607,708 the information on the relevant EEG methodology was described in non provisional patent application No. 13/341,465. In the summary of said non provisional patent application we talked about EEG correlations in brain areas during activation. In figures we showed a schematic representation of EEG channels locations, graphic representation showing healthy control brain activity regions using the International naming convention, graphic representation showing correlation coefficients of EEG signal (channel 13) of healthy control subject as a function of two time intervals. Then we discussed an approach for synchronizing recordings of EEG and a functional activity. In Functional Implementation section we described EEG signals with correlations over time intervals. We described how we did EEG recording in Laboratory Setup. In Software and Algorithms section we described the approach to EEG functional analysis, which we further explained in this patent application specifically for different types of stimulations, such as sound, light, etc. We also referenced the analysis of EEG signals in Claims section.

### SUMMARY OF THE INVENTION

It is the object of the present invention to provide cost-effective, EEG-based multi-functional imaging technology for monitoring human cortex and brain activities with the characteristic time window of about 10 milliseconds, which is comparable to the characteristic time of cortical modulations during sensory responses and motor activities.

### BRIEF DESCRIPTION OF DRAWINGS

The above, and other objects, features and advantages of the present invention will become apparent from the following description read in conjunction with the accompanying drawing:

FIG. 1 illustrates a schematics of the functional EEG imager. Here a stimulus (light/sound) (103) initiates brain processing (visual/auditory) of the subject (101) which is reflected in EEG changes recorded by a standard EEG recording system (104). Computer (102) algorithm divides the EEG records into trials, i.e., the epochs during which the subject performs repetitive identical actions triggered by sensory stimuli.

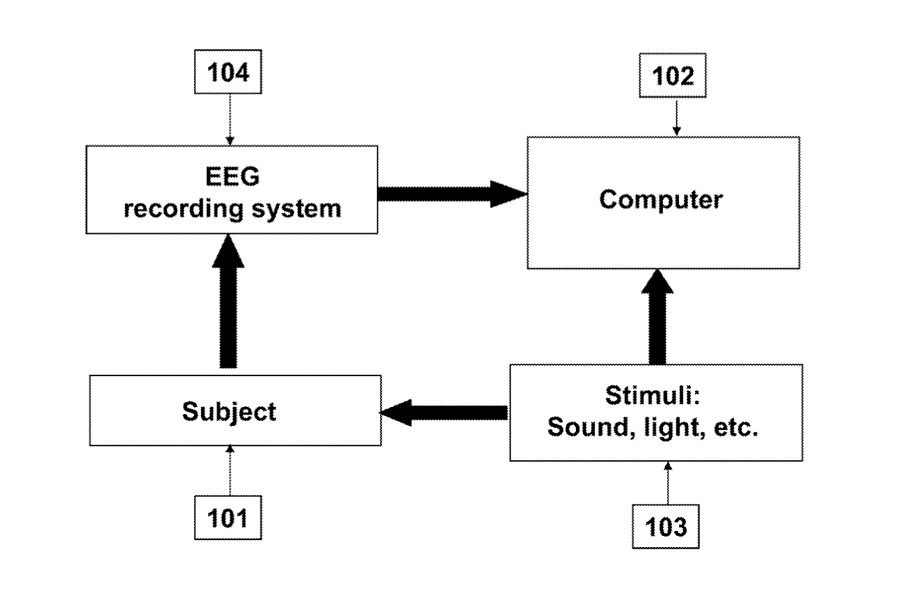

Further scope of applicability of the present invention will become apparent from the detailed description given hereafter. However, it should be understood that the detailed descriptions and specific examples, while including the preferred embodiments of the invention, are given by way of illustration only, since various changes and modifications within the spirit and scope of the invention will become apparent to those skilled in the art from this detailed description.

### DETAILED DESCRIPTION OF THE PRESENT INVENTION

In one of preferred embodiments, the impulse light/sound source is governed by the computer which sends command pulses to the source and creates simultaneously markers in the recording file. The markers are used to precisely slice the whole EEG session into well-aligned (with respect to each other) trials corresponding to the epochs during which the subject cortex or brain reacts to a single light/sound pulse stimulus. At the light/sound pulse duration of 1-2 second, and with the time interval between pulses of 1-2 second, the whole session duration with 100 trials is about 200-400 seconds, This preparation of a set of trials from the whole EEG session is a crucial point for further statistical analyses of cortical/brain activity. The total number of trials is determined by the desired width of the confidence interval for the correlation functions and should be determined experimentally.

The characteristic switching time of light-emitting diodes, which can be used as a light source, lies on the nanosecond time scale. Therefore the minimal size of time window of the imager will be restricted by the computer operating system delay only, which is under 10 ms. That should allow one to study the cortical/brain activity in response to the light stimulus with the time window of 10 millisecond, which is comparable to the characteristic time of neural modulations. For auditory stimulation, a sound source incorporated in conventional computer systems can be used. Thus, the characteristic time window of several seconds of fMRI technology is shortened in the proposed imaging, technology by about 3 orders of magnitude that enables to study rapid changes in cortex/brain activities.

In the other preferred embodiments, a repetition of light/sound stimuli is introduced inside each trail. In other words, each trial will contain a few stimuli (say, 2-5) with a fixed duration of each light/sound pulse and fixed time interval between them. The EEG/EEG correlation functions, computed with a statistical ensemble of such multi-stimulus trials, between EEG signals recorded from areas of the cortex or brain activated by such stimuli, should also demonstrate the analogous repetition in their time behavior. That allows one to determine precisely which areas of the cortex or brain are associated with activities such as hearing, vision, and motor activity, such as writing.

It is also possible to collect large amounts of data from different subjects in order to establish a range of correlation between brain areas that establish a representative sample of the general population. Individual subjects can be compared to the representative sample of the general population in order to identify different brain correlations in the individual subject compared to the general population. Such procedures and methods may be used to identify the regions of the brain that are responsible for the different correlations between the individual and the general population and how an individual subject's brain may vary from the general population.

In a similar manner, such comparisons can be performed between individual subjects and samples of selected populations with known neurological disorders. Those comparisons can be used to identify potential or actual neurological disorders in subjects where the EEG of the subjects corresponds to the EEG signals of the selective samples of populations with neurological disorders. Such comparisons are useful for early identification of illnesses which are potentially detectable by such comparisons.

The previous description of some embodiments is provided to enable any person skilled in the art to make or use the present technique. Various modifications to these embodiments will be readily apparent to those skilled in the art, and the generic principles defined herein may be applied to other embodiments without departing from the spirit or scope of the present disclosure. For example, one or more elements can be rearranged and/or combined, or additional elements may be added. Further, one or more of the embodiments can be implemented by hardware, software, firmware, middleware, microcode, or any combination thereof Thus, the present disclosure is not intended to be limited to the embodiments shown herein but is to be accorded the widest scope consistent with the principles and novel features disclosed herein.

Having described the technique in detail and by reference to the embodiments thereof, it will be apparent that modifications and variations are possible, including the addition of elements or the rearrangement or combination or one or more elements, without departing from the scope of the disclosure which is defined in the appended claims.

1. A system for detecting a functional connectivity between at least two regions of the brain using electroencephalography comprising:
a host computing device having a microprocessor, memory, input and output ports and a visual display;
in the computer memory, a stored set of EEG signal data derived from at least one subject responding to periodic and identical stimuli;
at least two EEG signal sensors coupled between a subject and the host computer, for detecting EEG signals generated by a subject while stimulated with the periodic, identical stimuli and while performing an identical response in each trial corresponding to each stimulus;
computing device input means for receiving an electronic signal corresponding to each stimulus;
computing, device input means for receiving EEG response signals from the subject who is subjected to the predetermined stimuli;
software means for storing temporal stimulus data representative of the time of occurrence of each stimulus;
software means for storing the time and the amplitude of subject EEG signals as EEG signal data in the computer memory
software means for processing the stored subject EEG signal data and the temporal stimulus data;
software means for generating graphic representations of the processed EEG signal data and temporal stimulus data for the a subject; and
software means for presenting on the visual display the graphic representations of the processed EEG signal data.

2. A method fir studying and observing functional time dependent connections between different regions of the brain using electroencephalography (PEG) comprising:
simultaneously recording at least two EEG signals in trials corresponding to periodic, identical responses of brain regions to periodic, identical stimuli:
recoding temporal stimulus data representative of the time of occurrence of each stimulus in each trial;
computing time dependent correlation functions between the recorded EEG signals;
and
indentifying a functional connectivity between different brain regions.

3. The system of claim 1 comprising a single EEG signal sensor for detecting time evolution of the activity of at least one region of the brain.

4. The system of claim 1 wherein stimuli are a set of a few sub-stimuli with fixed time interval between them in each trial.
